(19) United States
(12) Patent Application Publication
(10) Pub. No.: US 2025/0105384 A1
(43) Pub. Date: Mar. 27, 2025

(54) Systems, Methods, and Devices for Battery State Monitoring

(71) Applicant: TDK Corporation, Tokyo (JP)

(72) Inventors: Kei Hirata, San Jose, CA (US); Naoki Ota, Tokyo (JP)

(73) Assignee: TDK Corporation, Tokyo (JP)

(21) Appl. No.: 18/894,790

(22) Filed: Sep. 24, 2024

Related U.S. Application Data
(60) Provisional application No. 63/585,561, filed on Sep. 26, 2023.

(51) Int. Cl. H01M 10/48; G01N 33/00; H01M 10/0525; H01M 10/42

(52) U.S. Cl. CPC H01M 10/486; H01M 10/0525; H01M 10/482; G01N 33/005; H01M 2010/4271; H01M 2220/20

(57) ABSTRACT

An example battery system includes one or more battery packs. The battery system further includes a first sensor configured to detect a first property of the battery system and one or more second sensors configured to detect one or more second properties of the battery system. The battery system also includes control circuitry coupled to the one or more second sensors. The control circuitry is configured to detect a potential risk of thermal runaway of the one or more battery packs based on the detection of the one or more second properties, and, in response to detecting the potential risk of thermal runaway, activate the first sensor. The control circuitry is further configured to identify an increased risk of thermal runaway based on detection of the first property of the battery system, and, in response to identifying the increased risk of thermal runaway, disable the battery system.

### PRIORITY AND RELATED APPLICATIONS

This application claims priority to U.S. Provisional Patent Ser. No. 63/585,561, filed Sep. 26, 2023, entitled “Battery state monitoring sensors and its system,” which is incorporated by reference herein in its entirety.

### TECHNICAL FIELD

This application relates generally to battery systems, including but not limited to, stealth thermal runaway detection and selective use of gas sensors.

### BACKGROUND

The manufacture and use of battery electric vehicles (BEVs) is expanding globally. BEVs utilize high energy density batteries for increased mileage and driving power. For example, lithium (Li) ion batteries are often used for BEVs. Li ion batteries have high energy density but also have a risk of catching on fire, which is known as battery thermal runaway. Once battery thermal runaway happens, the battery catches fire, and the fire can be difficult to put it out due to the chemical characteristics of Li-ion batteries. Battery thermal runaway may be caused by product defects and/or external factors, such as an abnormal shock or water intrusion to the battery pack.

Therefore, thermal runaway detection and protection systems are important for battery systems, particularly in circumstances where consumers and users may be harmed, such as in BEVs. Some battery systems includes a battery management system (BMS) that monitors state of health (SoH) and/or state of charge (SoC) of battery system. A conventional BMS may include a current sensor, a voltage sensor, and/or a temperature sensor. However, these sensors cannot detect some battery abnormalities and/or properties that indicate a risk of thermal runaway. For example, battery abnormalities due to water intrusion into, and/or external shock on, the battery.

### SUMMARY

The present disclosure describes amongst other things, a thermal runaway detection system (e.g., an early detection system) for a battery system (e.g., having one or more battery modules and/or cells). Some of the disclosed systems include a notification/alarm component that can notify a user (e.g., a driver or passenger of a BEV) of a potential thermal runaway situation. The thermal runaway detection system may be integrated with a BMS to provide additional sensing, notification, and/or protection mechanisms. In some embodiments, the thermal runaway detection system includes multiple sensors. In some embodiments, the thermal runaway detection system uses data from multiple sensors to diagnose the state of thermal runaway. For example, the system may use a machine learning algorithm to analyze the data from the multiple sensors. Detecting a risk of thermal runaway at an earlier stage enables the system to alert users earlier, disable the batteries earlier, and/or take other remedial actions before the thermal runaway condition occurs (e.g., before anything catches fire). In some embodiments, the thermal runaway detection system is configured to activate one or more additional sensors in response to a first set of one or more sensors indicating an increased risk of thermal runaway. The one or more additional sensors may be high powered, have limited lifespan, and/or difficult to disable after activation. Selectively activating the additional sensor(s) in this manner can reduce power usage and extend sensor life.

In accordance with some embodiments, a battery system includes: (i) one or more battery packs; (ii) a first sensor configured to detect a first property of the battery system; (iii) one or more second sensors coupled to the one or more battery packs and configured to detect one or more second properties of the battery system; and (iv) control circuitry coupled to the one or more second sensors and configured to: (a) detect potential risk of thermal runaway of the one or more battery packs based on the detection of the one or more second properties; (b) in response to detecting the potential risk of thermal runaway, activate the first sensor; (c) identify an increased risk of thermal runaway based on detection of the first property of the battery system; and (d) in response to identifying the increased risk of thermal runaway, disable the battery system.

In accordance with some embodiments, a battery module includes: (i) one or more battery cells; (ii) a first sensor configured to detect a first property of the battery module; and (iii) one or more second sensors coupled to the one or more battery cells of the battery module and configured to detect one or more second properties of the battery module, where the first sensor is selectively activated based on data from the one or more second sensors.

In some embodiments, a battery system includes: (i) two or more of the battery modules; and (ii) control circuitry configured to: (a) identify a risk of thermal runaway in a first battery module of the two or more battery modules based on data from the respective one or more second sensors of the first battery module; (b) in response to identifying the risk of thermal runaway, activate the respective first sensor of the first battery module; (c) identify an increased risk of thermal runaway based on data from the respective first sensor; and (d) in response to identifying the increased risk of thermal runaway, disable the first battery module.

Thus, devices and systems are disclosed with methods for detecting and responding to thermal runaway indicators in a battery system. Such methods, devices, and systems may complement or replace conventional methods, devices, and systems for addressing thermal runaway in battery systems.

The features and advantages described in the specification are not necessarily all-inclusive and, in particular, some additional features and advantages will be apparent to one of ordinary skill in the art in view of the drawings, specification, and claims provided in this disclosure. Moreover, it should be noted that the language used in the specification has been principally selected for readability and instructional purposes and has not necessarily been selected to delineate or circumscribe the subject matter described herein.

### BRIEF DESCRIPTION OF THE DRAWINGS

So that the present disclosure can be understood in greater detail, a more particular description can be had by reference to the features of various embodiments, some of which are illustrated in the appended drawings. The appended drawings, however, merely illustrate pertinent features of the present disclosure and are therefore not necessarily to be considered limiting, for the description can admit to other effective features as the person of skill in this art will appreciate upon reading this disclosure.

FIG. 1A illustrates an example battery system in accordance with some embodiments.

FIG. 1B illustrates an example thermal runaway monitoring system and flow in accordance with some embodiments.

FIG. 2A illustrates an example chart of building for thermal runaway in accordance with some embodiments.

FIG. 2B illustrates an example monitoring system and flow for monitoring for thermal runaway in accordance with some embodiments.

FIG. 3 is a flow diagram illustrating an example method of identifying risk of thermal runaway in accordance with some embodiments.

FIGS. 4A-4C illustrate example sensor data corresponding to various battery events in accordance with some embodiments.

In accordance with common practice, the various features illustrated in the drawings are not necessarily drawn to scale, and like reference numerals can be used to denote like features throughout the specification and figures.

### DESCRIPTION OF EMBODIMENTS

As discussed above, conventional BMSs include a few sensors such as current, voltage, and temperature sensors. However, these sensors may be unable to detect (or too late in detecting) a potential thermal runaway condition. The present disclosure describes monitoring systems (e.g., a sub-system of a BMS) to monitor battery components (e.g., a battery module, pack, or cell) using multiple sensors and sensor data analysis techniques. In some embodiments, the monitoring system includes a variety of sensors, including sensors not present in conventional BMSs. In some embodiments, the monitoring system includes one or more high power sensors that are selectively enabled based on data from one or more low power sensors. These monitoring systems can detect potential thermal runaway conditions earlier and/or more accurately. Additionally, these systems may provide more battery monitoring redundancy for increased security and fault tolerance.

In accordance with some embodiments, a monitoring system described herein is configured to provide two-step thermal runaway protection. In a first step, a battery abnormality is detected, the abnormality having the potential to cause thermal runway within the battery. For example, the abnormality may be due to water intrusion or external shock on a battery pack or module. As an example, water can intrude into a BEV battery module due to heavy rain or flooding, and external shock to battery pack may be caused by car crash or a debris hit to battery pack. In some embodiments, the monitoring system alerts a user of the battery abnormality (e.g., by generating a visual or audio notification).

In the second step, build up to a thermal runaway condition is detected (e.g., earlier than in conventional systems). For example, build up to the thermal runaway can include the battery component heating up and combusting oxygen (e.g., dioxygen (O2)) and carbon from anode and cathode material of the battery component. The heat and carbon combustion can cause carbon monoxide (CO) (and/or carbon dioxide (CO2)) emissions. The data from rising CO and/or CO2 gas concentration (e.g., detected by a gas sensor) and rising temperature (e.g., detected by a temperature sensor) may be analyzed to determine whether a thermal runaway condition is building in the battery component. In addition, or alternatively, pressure sensor data may be used to determine the buildup of the thermal runaway condition. In some embodiments, the monitoring system alerts a user to the buildup of the thermal runaway condition (e.g., by generating a visual or audio notification). In some embodiments, the monitoring system disables the battery component and/or takes other remedial action (e.g., venting and/or cooling) in response to detecting the buildup of the thermal runaway condition.

As described in detail below, a monitoring system of the present disclosure may include one or more of the following sensor types: a strain sensor, a hydrogen (H2) gas sensor, a pressure sensor, a temperature sensor, a carbon-oxygen gas (e.g., CO and/or CO2) sensor, an electric current sensor, and an accelerometer. Each sensor may measure a different aspect/property of the battery component and an embedded system algorithm may analyze the data from the various sensors and determine a battery state and/or a risk of thermal runaway. For example, the strain sensor, temperature sensor and hydrogen gas sensor may be used for detection of electrolysis due to water intrusion, which can trigger thermal runaway. As another example, the pressure sensor and accelerometer may be used for detection of external shock on the battery component (e.g., caused by a car crash or impact of debris). As another example, the temperature sensor and current sensor may be used for detection of excessive heating and/or overcharging of the battery component.

As mentioned above, individual sensor data may be insufficient to judge whether the battery component is at risk of thermal runway. Therefore, some embodiments include the integration of data from multiple sensor types to diagnose a battery state and/or buildup of a thermal runaway condition. In some embodiments, the monitoring system logs the sensor data and analyses changes over time (e.g., trends) in the data to identify battery abnormalities and/or buildup of a thermal runaway condition.

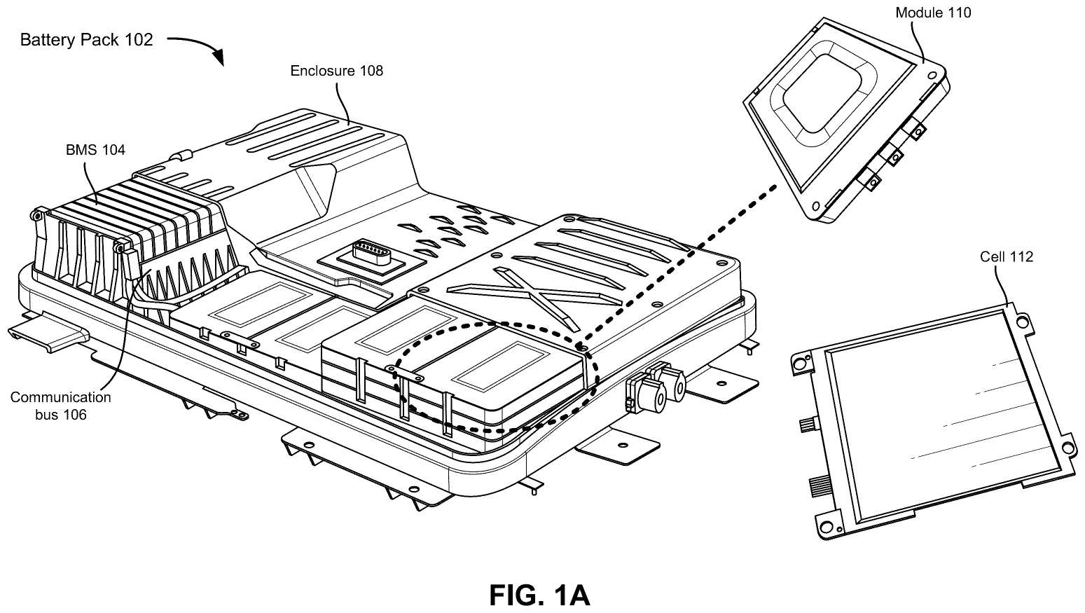

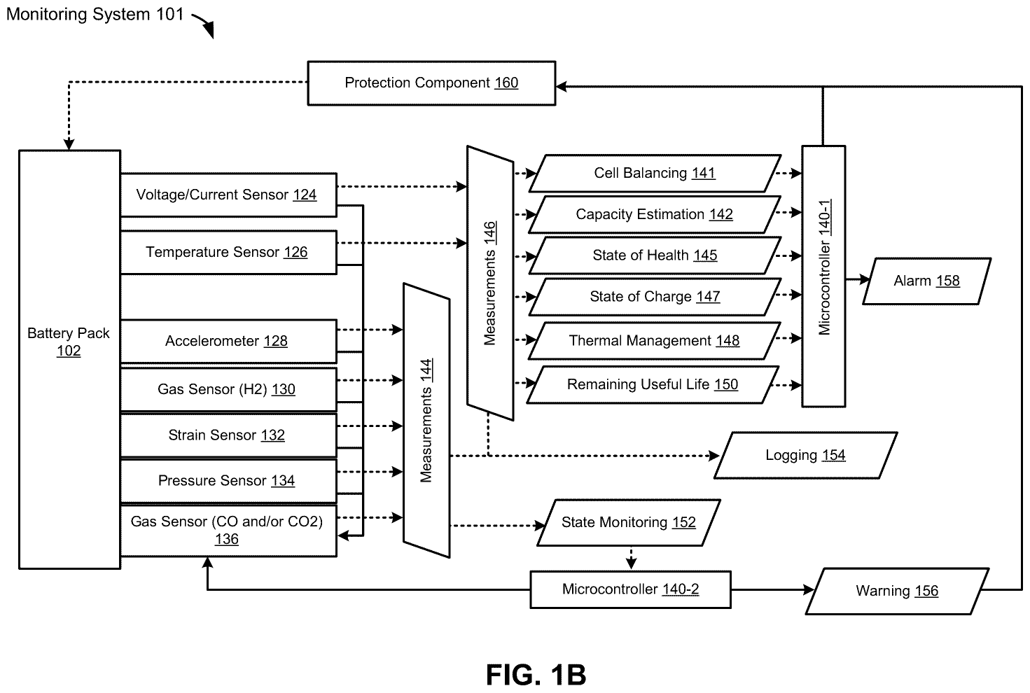

FIG. 1A illustrates a battery system that includes a battery pack 102 in accordance with some embodiments. In some embodiments, the battery system includes multiple battery packs. The battery pack 102 includes multiple battery modules 110 (e.g., a cluster of battery modules) and a BMS 104 coupled to the battery modules 110 via a communication bus 106. In the example of FIG. 1A, the battery pack 102 includes an enclosure 108 (e.g., a mechanical enclosure). Each battery module 110 may include one or more battery cells 112 (e.g., may contain a cluster of battery cells). Each battery cell may be a pouch type, prismatic type, or cylindrical type.

FIG. 1B illustrates a thermal runaway monitoring system 101 and flow in accordance with some embodiments. In FIG. 1B, the battery pack 102 is coupled with a voltage/current sensor 124, a temperature sensor 126, an accelerometer 128, a hydrogen gas sensor 130, a strain sensor 132, a pressure sensor 134, and a carbon monoxide (and/or carbon dioxide) gas sensor 136. In some embodiments, the battery pack 102 is coupled to a subset, or superset, of the sensors shown in FIG. 1B. In some embodiments, the battery pack 102 is coupled to a carbon dioxide sensor and to a carbon monoxide sensor. In some embodiments, the voltage/current sensor 124 and the temperature sensor 126 correspond to a battery monitoring system. Measurements 146 from the voltage/current sensor 124 and/or the temperature sensor 126 are used to determine cell balancing 141, capacity estimation 142, state of health 145, state of charge 147, thermal management 148, and/or remaining useful life 150. In accordance with some embodiments, a microcontroller 140-1 performs the above operations based on the measurements 146 from the voltage/current sensor 124 and/or the temperature sensor 126. In accordance with a determination that one or more of the above operations indicates an abnormal state for the battery pack 102 or nearing end of life for the battery pack 102, the microcontroller 140-1 generates an alarm 158 (e.g., a visual and/or audio notification). For example, if data from one or more of the sensors is outside of a baseline operating range, the microcontroller 140-1 may generate the alarm 158. In accordance with a determination that the battery pack 102 is in an abnormal state, the microcontroller 140-1 may shutdown, power down, disconnect, or otherwise disable the battery pack 102 using the protection component 160.

Measurements 144 from the accelerometer 128, the hydrogen gas sensor 130, the strain sensor 132, the pressure sensor 134, and/or the CO and/or CO2 gas sensor 136 are used for state monitoring 152. In accordance with some embodiments, the measurements 144 are used with the measurements 146 to determine cell balancing 141, capacity estimation 142, state of health 145, state of charge 147, thermal management, and/or remaining useful life 150. In accordance with some embodiments, the measurements 144 and/or the measurements 146 are stored via logging 154. In some embodiments, the stored measurements are used to identify changes in attributes of the battery pack 102 over time. In accordance with some embodiments, a microcontroller 140-2 performs the state monitoring 152. In some embodiments, the microcontroller 140-1 is the same as the microcontroller 140-2. In some embodiments, the microcontroller 140-1 is separate and distinct from the microcontroller 140-2. In some embodiments, at least one of the microcontrollers 140 is replaced with a controller or other type of control circuitry.

The microcontroller 140-2 generates a warning 156 (e.g., a visual and/or audio notification) in accordance with a determination that the measurements 144 (and optionally the measurements 146) indicate a battery pack abnormality (such as buildup of a thermal runaway condition). In accordance with a determination that the battery pack 102 is in an abnormal state (e.g., that there is an increased risk of thermal runaway), the microcontroller 140-2 may shutdown, power down, disconnect, or otherwise disable the battery pack 102 using the protection component 160.

In some embodiments, the gas sensor 136 is maintained in an off state (or a low power state) until activated by the microcontroller 140-2. In some embodiments, the microcontroller 140-2 activates the gas sensor 136 in accordance with a determination that the battery pack 102 is in an abnormal state. In some embodiments, after activating the gas sensor 136, the microcontroller 140-2 analyzes data from the gas sensor 136 to determine whether remedial action needs to be taken to prevent a thermal runaway. For example, the remedial action may be to vent, cool, shutdown, power down, disconnect, and/or otherwise disable the battery pack 102 using the protection component 160. In some embodiments, the data from the CO and/or CO2 gas sensor 136 is analyzed with data from one or more of the other sensors to determine whether the remedial action is to be taken.

As shown in FIG. 1B, example sensors for the monitoring system 101 include a temperature sensor, a strain sensor, a current sensor, a pressure sensor, a gas sensor and an accelerometer. The temperature sensor 126 may be a negative thermal coefficient (NTC) temperature sensor or a positive thermal coefficient (PTC) temperature sensor. The strain sensor 132 may be composed of resistive material such as Cr—N, Cr—Al—N, Ni—Cr, or Cu—Ni. The hydrogen gas sensor 130 may be a thermistor type or a metal oxide type gas sensor. A thermistor type gas sensor may include a heater and a NTC thin film. For a thermistor type gas sensor, gas generation can be detected by a gas thermal conductivity difference from air or other normal (e.g., baseline) state. For a thermistor type gas sensor with an NTC thin film, electrical resistance changes due to a flowing gas on the NTC thin film. A metal oxide type gas sensor may include a heater and a metal oxide material (e.g., a powder). In this type of sensor, once gas absorbs on the metal oxide material, the electric resistance changes due to surface electrical potential change caused by the gas.

The voltage/current sensor 124 may be a shunt type, magnetic hall type, or xMR (e.g., a giant magnet resistance, an anisotropic magneto resistance, or a tunneling magnet resistance) type sensor. The pressure sensor 134 may include a piezoelectric material such as AlN, BaTiO, Bithmus system, PbZrTiO, or impurity doped Si type. The accelerometer 128 may be a micro-electro-mechanical systems (MEMS) capacitive type accelerometer or a piezoelectric type accelerometer. In some embodiments, the accelerometer 128 detects acceleration along at least one axis. In some embodiments, the accelerometer 128 detects acceleration along three axes.

In some embodiments, one or more of the sensors for the monitoring system 101 are attached inside a battery pack and/or inside a battery module. In some embodiments, one or more of the sensors for the monitoring system 101 are attached outside of a battery module and/or a battery pack. For example, the sensors may be attached at any appropriate position inside or outside of battery pack or battery module. As a specific example, the strain sensor 132, the temperature sensor 126, the voltage/current sensor 124, and the accelerometer 128 may be attached on each battery module or battery pack, and the pressure sensor 134, the hydrogen gas sensor 130, and the CO and/or CO2 gas sensor 136 may be attached inside of a battery pack or battery module.

In some embodiments, data from the hydrogen gas sensor 130, the strain sensor 132, and the temperature sensor 126 is used to diagnose battery abnormality (e.g., to detect the battery abnormality due to water intrusion). When water intrudes into a battery pack, electrolysis can occur due to water bridging between battery cells. Electrolysis can cause corrosion and deterioration of the battery outer cover and/or separators due to a high electrical potential gap between battery cell, which causes an electrical short between the cathode and the anode. Once an electrical short occurs, the battery cell may immediately begin to heat.

Battery cell corrosion may be detected based on an electrical potential between a battery cell and a battery pack (e.g., by monitoring voltage between a battery cell and a battery pack). For example, the electrical potential can indicate a battery cell deterioration.

External shock may be detected using data from a pressure sensor and/or an accelerometer. For example, the pressure sensor detects changes in pressure inside of a battery pack/module. The change in pressure may indicate breakage and/or deformation of the case of the battery pack/module. Additionally, an accelerometer is able to detect an external shock (that could cause damage to the battery pack/module), which may be correlated with the change in pressure to indicate that the change in pressure is due to the external shock.

Water electrolysis causes H2 and O2 gas emissions, thus the pressure and H2 concentration inside the battery module and/or battery pack increases. In addition, water electrolysis may cause the battery module/pack to deform. An H2 gas sensor (e.g., the hydrogen gas sensor 130) can be used to detect the H2 gas generation and a pressure sensor (e.g., the pressure sensor 134) can be used to detect the increased pressure in the battery pack/module. Additionally, a strain sensor (e.g., the strain sensor 132) can detect a deformation on the battery pack/module due to the water electrolysis (e.g., the strain sensor is capable of detecting even small deformations). A temperature sensor (e.g., the temperature sensor 126) can monitor a battery pack/module or a particular region of the battery pack/module to detect the heating due to electrolysis. Additionally, a battery abnormality due to electrolysis can be detected via relative changes of temperature and strain (e.g., because temperature and strain normally correlate by thermal expansion of material). In particular, an electrolysis process can cause a battery pack/module to expand/inflate without a corresponding temperature rise, which disrupts the correlation between temperature and strain. This data may be combined with data from the H2 gas sensor and/or pressure sensor that may indicate a corresponding increase in gas inside of the battery pack/module. In this way, the battery state diagnosis may be performed (e.g., by an embedded algorithm) by analyzing information from multiple sensors (e.g., detecting relative changes and/or correlations).

In some embodiments, the monitoring system 101 (e.g., one of the microcontrollers 140) identifies the battery abnormality and a potential cause (e.g., corrosion, external shock, and/or water electrolysis) and generates an alert that indicates the potential cause. In some embodiments, the alert indicates the potential cause and one or more recommended remedial actions for the user (e.g., evacuate the area, disable the device using the battery pack, contact emergency services, and/or schedule maintenance for the battery pack).

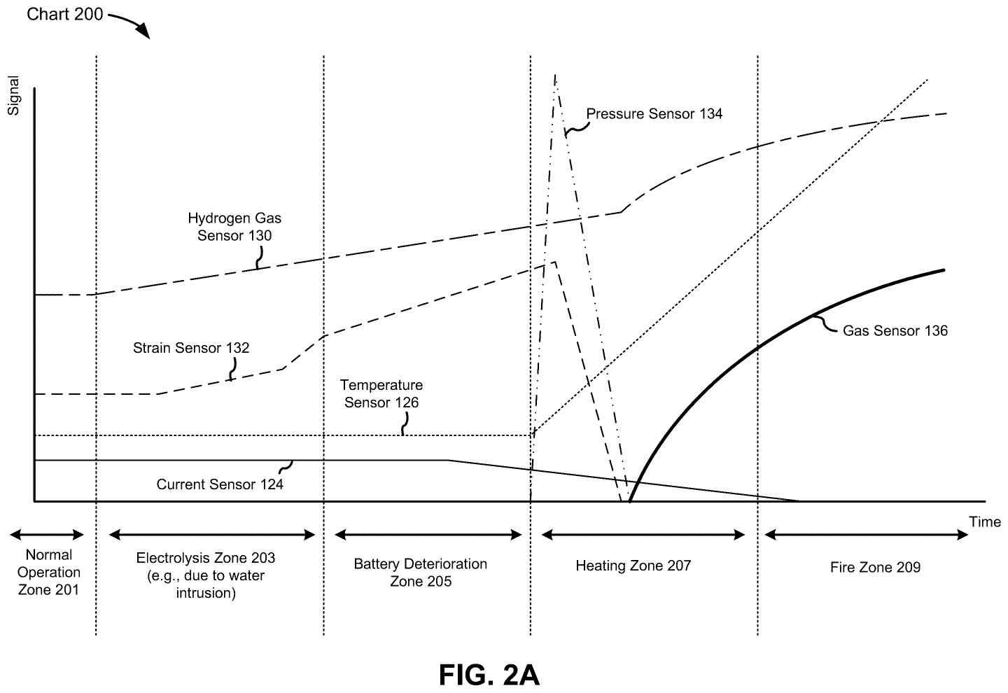

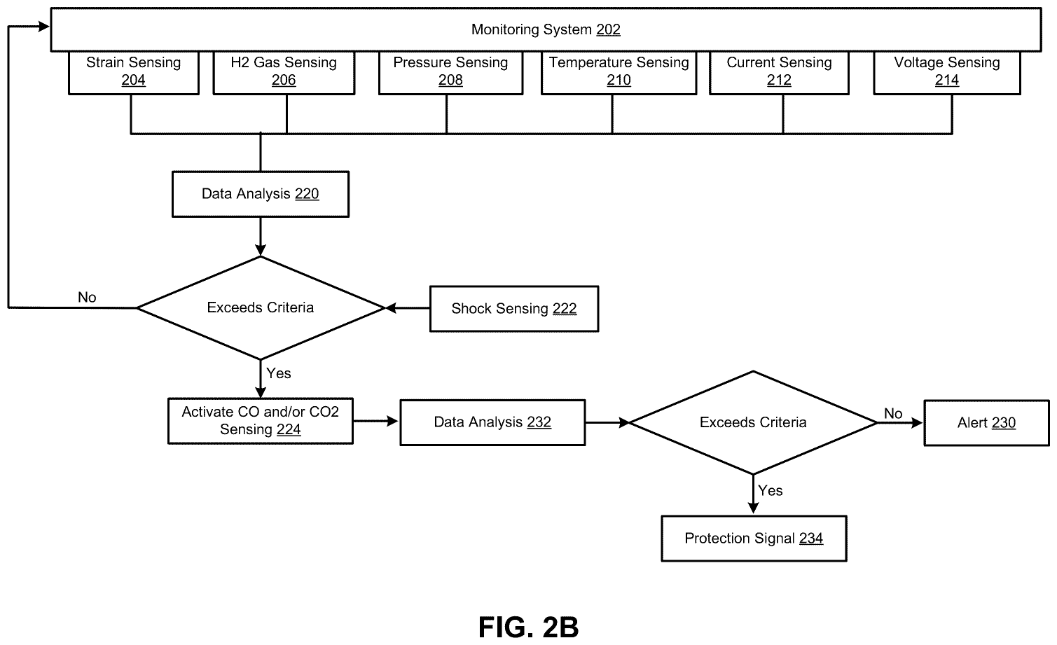

FIG. 2A illustrates a chart 200 of buildup for thermal runaway in accordance with some embodiments. The chart 200 is split into different zones that indicate what is occurring at a battery component (e.g., the battery pack 102, the battery module 110, or the battery cell 112). The zones include a normal operation zone 201, an electrolysis zone 203, a battery deterioration zone 205, a heating zone 207, and a fire zone 209. The normal operation zone 201 corresponds to normal (proper) operation of the battery component. In the normal operation zone 201, the hydrogen gas sensor 130, the strain sensor 132, the temperature sensor 126, and the current sensor 124 indicate that the corresponding properties of the battery component are constant (e.g., the battery component is in a steady state). In some embodiments, the data from the sensors fluctuates within a range of values that correspond to normal operation of the battery component. The electrolysis zone 203 water electrolysis occurring within the battery component (as described above). In the electrolysis zone 203, the hydrogen gas sensor 130 indicates an increase in hydrogen gas and the strain sensor 132 indicates that battery deformation is occurring as a result of the electrolysis. Notably, the temperature sensor 126 and the current sensor 124 may not detect any change during the electrolysis. The battery deterioration zone 205 corresponds to deterioration of the battery component (e.g., electrolyte leakage) due to the electrolysis. In the battery deterioration zone 205, the hydrogen gas sensor 130 indicates an additional increase in hydrogen gas and the strain sensor 132 indicates further deformation of the battery component. Additionally, the current sensor 124 indicates a decrease in current due to the battery deterioration. As shown in FIG. 2A, detecting electrolysis (e.g., within the electrolysis zone 203) allows for the battery component to be disabled (e.g., powered down and/or disconnected) before battery deterioration occurs. The electrolysis zone 203 and the battery deterioration zone correspond to a stealth (cold) stage of the buildup of thermal runaway. For example, the stealth stage occurs prior to heating of the battery component.

The heating zone 207 corresponds to the battery component heating up (e.g., and combustion of a battery component anode). In the heating zone, the strain sensor 132 and the pressure sensor 134 indicate a further deformation of the battery and corresponding increase in internal pressure until a vent occurs (e.g., the battery component casing ruptures), after which, the deformation and pressure decreases. The current sensor 124 indicates a further decrease in current flow while the temperature sensor 126 indicates an increase in temperature and the hydrogen gas sensor 130 indicates the presence of additional H2 gas. In some embodiments, the CO and/or CO2 gas sensor 136 is enabled in the battery deterioration zone 205 or the heating zone 207 (e.g., in response to detecting the increases in strain and/or hydrogen gas). The CO and/or CO2 gas sensor 136 detects an increase in CO and/or CO2 during the heating zone 207 (e.g., after the vent occurs in the battery component). The fire zone 209 corresponds to a fire in at least a portion of the battery component (e.g., a thermal runaway condition). In the fire zone 209, the temperature increases along with the presence of CO and/or CO2 while the current diminishes, and additional hydrogen gas may be present. In some embodiments, an alert is generated (and/or other remedial action is taken) in response to detecting the increase in hydrogen gas and/or strain during the electrolysis zone 203. In some embodiments, an alert is generated (and/or other remedial action is taken) in response to detecting the increase in hydrogen gas, the increase in strain, and/or the decrease in current during the battery deterioration zone 205. In some embodiments, an alert is generated (and/or other remedial action is taken) in response to detecting the increase in hydrogen gas, strain, temperature, CO and/or CO2 gas, and/or pressure during the heating zone 205.

FIG. 2B illustrates a monitoring system 202 and flow for monitoring for thermal runaway in accordance with some embodiments. The monitoring system 202 includes strain sensing 204 (e.g., corresponding to the strain sensor 132), H2 gas sensing 206 (e.g., corresponding to the gas sensor 130), pressure sensing 208 (e.g., corresponding to the pressure sensor 134), temperature sensing 210 (e.g., corresponding to the temperature sensor 126), current sensing 212 (e.g., corresponding to the voltage/current sensor 124), voltage sensing 214 (e.g., corresponding to the voltage/current sensor 124), and shock sensing 222 (e.g., corresponding to the accelerometer 128) for a battery component. In some embodiments, the monitoring system 202 includes a subset or superset of the sensing types shown in FIG. 2B. For example, the monitoring system 202 may include only one of the current sensing and voltage sensing. The monitoring system 202 performs a data analysis 220 of the various sensing data (e.g., using a microcontroller or other type of control circuitry).

In accordance with a determination that the analyzed data exceeds one or more criteria (e.g., is not within a threshold range of a baseline value), the monitoring system 202 activates CO (and/or CO2) sensing 224 (e.g., the gas sensor 136 is activated). For example, additional power may be supplied to the CO and/or CO2 gas sensor and/or data from the CO and/or CO2 sensor may be stored and/or analyzed in response to being activated. In some embodiments, in accordance with the determination that the analyzed data exceeds the one or more criteria, the monitoring system 202 generates an alert. The monitoring system 202 performs a data analysis 232 on the CO and/or CO2 sensing 224 (and optionally on the other sensing described above). In accordance with a determination that the analyzed CO and/or CO2 sensing data exceeds one or more criteria, the monitoring system 202 generates a protection signal 234 (e.g., which is transmitted to the protection component 160) to disable (e.g., power down and/or disconnect) the battery component. In some embodiments, in accordance with the determination that the analyzed CO and/or CO2 sensing data exceeds the one or more criteria, the monitoring system 202 generates an alert indicating the presence of excess CO and/or CO2 gas. In accordance with a determination that the analyzed CO and/or CO2 sensing data does not exceed the one or more criteria, the monitoring system 202 may generate an alert 230 (e.g., indicating that a battery abnormality has been detected).

In some embodiments, sensor data is analyzed by a system algorithm (e.g., a machine-learning (ML) algorithm) to determine if the sensor data meets one or more criteria for normal operation. In some embodiments, the one or more criteria for normal operation are preset for (and/or learned by) the monitoring system. In some embodiments, the one or more criteria for normal operation are based on environmental and known factors. In some embodiments, the monitoring system identifies a battery abnormality in response to the sensor data not meeting the one or more criteria for normal operation. As discussed above, in some embodiments, the monitoring system activates CO and/or CO2 sensing and/or battery protections in response to identifying a battery abnormality.

In some embodiments, a system algorithm determines a battery state for the battery component based on data from multiple types of sensors (e.g., current, voltage, temperature, H2 gas concentration, acceleration, and/or pressure sensors). The battery state may be one of: normal state, electrolysis state, deterioration state, heating state, or fire state. In some embodiments the system algorithm monitors/analyzes the correlations and deviations between various battery parameters, such as temperature, pressure, strain, gas concentration, and/or current. In some embodiments, the system algorithm identifies a battery abnormality based on correlation/deviation data. In some embodiments, the monitoring system stores sensor data and compares current sensor data with past sensor data (e.g., to identify trends and other changes over time). In some embodiments, the system algorithm identifies a battery abnormality based on a change in sensor data over time (and/or a rate of change of sensor data over time). For example, the system algorithm may identify a battery abnormality based on a correlation parameter changing over time. In another example, the system algorithm monitors the values of physical parameters (such as temperature, strain, gas concentration, current, and pressure) and identifies battery abnormalities based on individual parameters and/or parameter combinations. As a specific example, a pressure sensor and accelerometer may be used to monitor external shocks, and, in response to detecting an external shock, the system algorithm activates a CO and/or CO2 gas sensor to check the occurrence of thermal runway (or the building to a thermal runaway). In response to detecting that the CO and/or CO2 gas concentration exceeds the one or more criteria, the system algorithm generates an alert and/or activates battery protections.

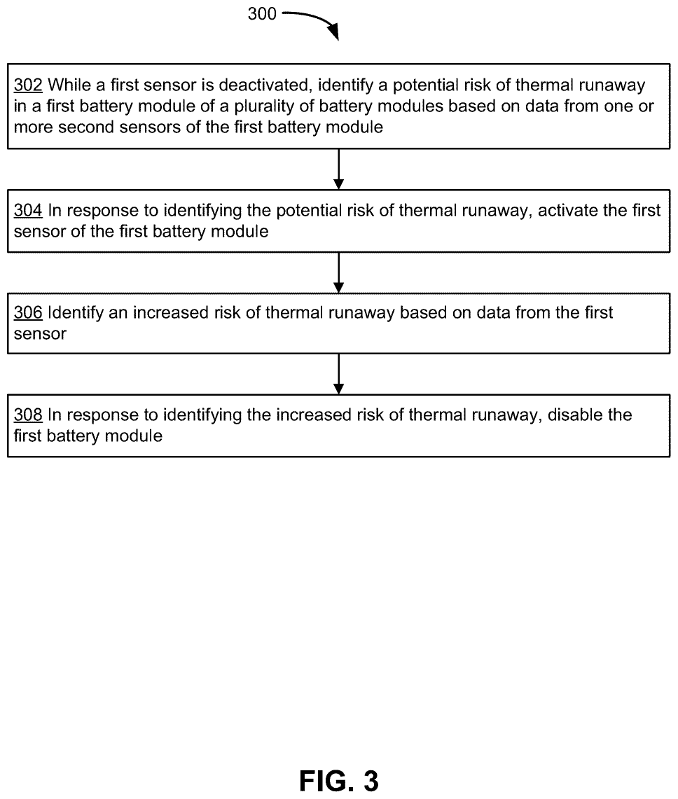

FIG. 3 is a flow diagram illustrating a method 300 of identifying risk of thermal runaway in accordance with some embodiments. The method 300 may be performed at a computing system (e.g., the monitoring system 101 or the monitoring system 202) having control circuitry (e.g., the microcontrollers 140) and memory storing instructions for execution by the control circuitry. In some embodiments, the method 300 is performed by executing instructions stored in the memory of the computing system.

While a first sensor is disabled (e.g., a CO and/or CO2 sensor), the system identifies (302) a potential risk of thermal runaway in a first battery module (e.g., the battery module 110) of a plurality of battery modules based on data from one or more second sensors of the first battery module. In some embodiments, the one or more second sensors include a voltage sensor, a current sensor, a temperature sensor, an accelerometer, a hydrogen gas sensor, a strain sensor, and/or a pressure sensor. In some embodiments, the one or more second sensors are attached to an interior or exterior of the first battery module. In some embodiments, each battery module in the plurality of battery modules includes a respective set of sensors.

In response to identifying the potential risk of thermal runaway, the system activates (304) the first sensor (e.g., the gas sensor 136) of the first battery module. In some embodiments, the first sensor is a CO and/or CO2 gas sensor. In some embodiments, the first sensor is a high-power sensor (e.g., requiring higher power than the one or more sensors of the first battery module). In some embodiments, in response to identifying the potential risk of thermal runaway, the system generates a first alert (e.g., a visual and/or audio notification that indicates the risk).

The system identifies (306) an increased risk of thermal runaway based on data from the first sensor. In some embodiments, the system identifies the increased risk of thermal runaway based on data from the first sensor and data from the one or more second sensors (e.g., individually and/or in combination).

In response to identifying the increased risk of thermal runaway, the system disables (308) the first battery module. In some embodiments, in response to identifying the increased risk of thermal runaway, the system generates a second alert (e.g., a visual and/or audio notification that indicates the increased risk). In some embodiments, the first alert has one or more parameters that are different than the parameters of the second alert.

Although FIG. 3 illustrates a number of logical stages in a particular order, stages which are not order dependent may be reordered and other stages may be combined or broken out. Some reordering or other groupings not specifically mentioned will be apparent to those of ordinary skill in the art, so the ordering and groupings presented herein are not exhaustive. Moreover, it should be recognized that the stages could be implemented in hardware, firmware, software, or any combination thereof.

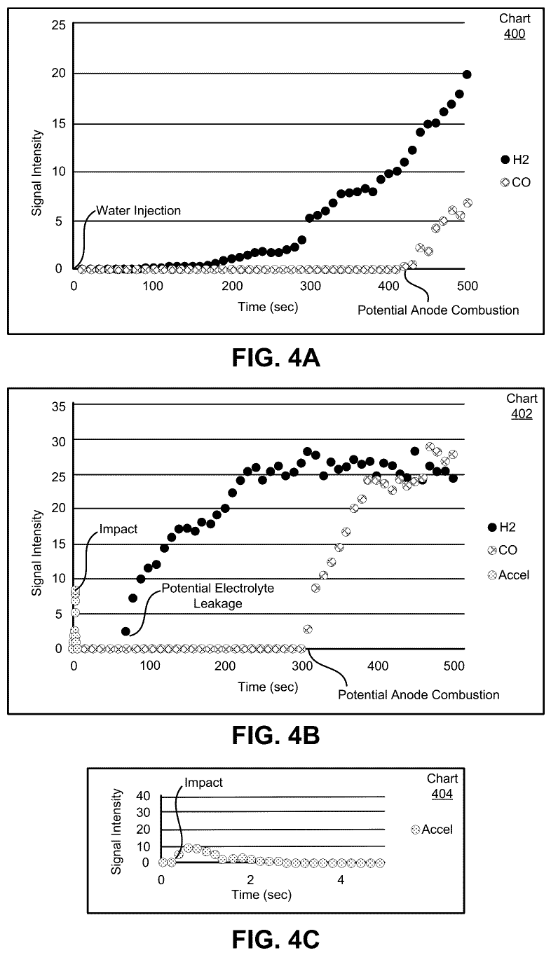

FIGS. 4A-4C illustrate example sensor data in a chart 400 corresponding to various battery events in accordance with some embodiments. FIG. 4A illustrates an example in which water (e.g., 10 milliliters) is injected into a battery pack (e.g., the battery pack 102) at time zero. In the example of FIG. 4A, an H2 gas sensor (e.g., the H2 gas sensor 130) and a CO gas sensor (e.g., the CO and/or CO2 gas sensor 136) are coupled to the battery pack. The vertical axis of the chart 400 corresponds to signal intensity (e.g., a unitless measure of % of maximum signal output). As an example, the chart 400 shows the H2 gas increasing around 180 seconds after the water injection event. In some embodiments, the H2 gas sensor detects the increase in H2 gas, and the system (e.g., a controller coupled to the H2 gas sensor) identifies the increase as a potential risk of thermal runaway. In the example of FIG. 4A, the CO sensor detects an increase in CO gas at around 420 seconds after the water injection event. The increase in CO gas may correspond to an anode combustion event in the battery pack. In some embodiments, the CO gas sensor detects the increase in CO gas, and the system (e.g., a controller coupled to the CO gas sensor) identifies the increase as an increased risk of thermal runaway.

FIGS. 4B and 4C illustrate an example in which a battery pack (e.g., the battery pack 102) suffers an impact (e.g., at around time 1 second). FIG. 4C illustrates a chart 404 showing data from an accelerometer (e.g., the accelerometer 128) during the first 4 seconds. The data from the accelerometer in FIG. 4C indicates an impact with the battery pack in the first two seconds. FIG. 4B illustrates a chart 402 that shows data from the accelerometer and corresponding data from H2 and CO gas sensors. As shown in FIG. 4B, the H2 gas begins to increase approximately 70 seconds after the impact. For example, the H2 gas increase may be due to electrolyte leakage from a battery cell of the battery pack. In some embodiments, the H2 gas sensor detects the increase in H2 gas, and the system (e.g., a controller coupled to the H2 gas sensor) identifies the increase as a potential risk of thermal runaway. The chart 402 further shows an increase in CO gas approximately 300 seconds after the impact. For example, the CO gas increase may be due to anode combustion within the battery pack. In some embodiments, the CO gas sensor detects the increase in CO gas, and the system (e.g., a controller coupled to the CO gas sensor) identifies the increase as an increased risk of thermal runaway. In some embodiments, a CO2 gas sensor is used to detect increases in CO2 gas (e.g., in addition to, or alternatively to, a CO gas sensor detecting the CO gas increase).

In light of the above disclosure, certain embodiments are described below.

In accordance with some embodiments, a monitoring system is configured to detect battery parameters and determine whether the battery is in a normal state or an abnormal state based on the detected parameters. The battery parameters are detected using multiple sensors. A system algorithm (e.g., an ML algorithm) may be used to determine the battery state based on the detected battery parameters. In some embodiments, the monitoring system includes one or more of: a strain sensor, an H2 gas sensor, a temperature sensor, a pressure sensor, a CO and/or CO2 gas sensor, a current sensor, a voltage sensor, and accelerometer. In some embodiments, the monitoring system includes an integrated circuit (IC) configured to control operation of the sensors, analyze the sensor data, determine the battery status, and perform remedial actions in response to determining an abnormal battery status. In this way, battery abnormalities may be detected, and remedial action may be taken, before thermal runaway occurs (e.g., before a fire occurs in the battery component).

(A1) In one aspect, some embodiments include a battery system that includes: (i) one or more battery packs (e.g., with each battery pack having one or more battery cells); (i) a first sensor (e.g., the gas sensor 136) configured to detect a first property of the battery system; (ii) one or more second sensors (e.g., one or more of the sensors 124, 126, 128, 130, 132, and 134) coupled to the one or more battery packs (e.g., the battery pack 102) and configured to detect one or more second properties of the battery system; and (iii) control circuitry (e.g., a microcontroller 140) coupled to the one or more second sensors and configured to: (a) detect a potential risk of thermal runaway of the one or more battery packs based on the detection of the one or more second properties (e.g., corresponding to the zone 203 and/or 205 in FIG. 2A); (b) in response to detecting the potential risk of thermal runaway, activate the first sensor (e.g., provide additional power to the first sensor); (c) identify an increased risk of thermal runaway based on detection of the first property of the battery system (e.g., corresponding to the zone 207 and/or the zone 209 in FIG. 2A); and (d) in response to identifying the increased risk of thermal runaway, disable the battery system (e.g., using the protection component 160). In some embodiments, the one or more battery packs comprise one or more of pouch-type cells, prismatic-type cells, and cylindrical-type cells. In some embodiments, the control circuitry comprises a microcontroller, a controller, a central processing unit (CPU), or other type of control circuitry. In some embodiments, the thermal runaway condition is based on combined data from the first sensor and the one or more second sensors. In some embodiments, the control circuitry is configured to log data from the one or more second sensors and/or the first sensor (e.g., corresponding to the logging 154). In some embodiments, disabling the battery system comprises disabling one or more of the one or more battery packs (e.g., disconnecting one or more terminals of the one or more battery packs). In some embodiments, detecting the potential risk of thermal runaway comprises detecting intrusion into one or more of the one or more battery packs (e.g., water intrusion).

(A2) In some embodiments of A1, the first sensor is a carbon monoxide (CO) sensor. In some embodiments, the first sensor is a carbon dioxide (CO2) sensor.

(A3) In some embodiments, of A1 or A2, the one or more second sensors comprise one or more of a hydrogen gas sensor, and a strain sensor. In some embodiments, the strain sensor includes a resistive material such as Cr—N, Cr—Al—N, Ni—Cr, Cu—Ni. In some embodiments, the hydrogen sensor is an H2 gas sensor. As an example, the H2 gas sensor may be a thermistor type sensor or a metal oxide type gas sensor. A thermistor type gas sensor may include a heater and a negative thermal coefficient (NTC) thin film. For example, gas generation may be detected via a gas thermal conductivity difference from air or normal state as electric resistance changes due to flowing gas on the NTC thin film. A metal oxide type gas sensor may include a heater and a metal oxide material. For example, once gas absorbs on the metal oxide material, the electric resistance changes by due to a surface electrical potential change.

(A4) In some embodiments of any of A1-A3: (i) the one or more second sensors comprises a plurality of different types of sensors; and (ii) the control circuitry is configured to detect the potential risk of thermal runaway using a machine learning algorithm to analyze data from the plurality of different types of sensors. In some embodiments, the control circuitry is configured to detect the potential risk of thermal runaway using one or more lookup tables and the data from the plurality of different types of sensors. In some embodiments, the ML algorithm is trained (e.g., fine-tuned) to analyze data from multiple types of battery sensors. In some embodiments, the ML algorithm comprises one or more neural networks, one or more transformers, one or more random forests, and/or other types of ML components.

(A5) In some embodiments of any of A1-A4, at least one of the one or more second sensors is positioned within a battery pack of the one or more battery packs. For example, the one or more second sensors may be attached to a position inside or outside of a battery pack or battery module. For example, a strain sensor, temperature sensor, current sensor, and/or accelerometer may be attached on each battery module or battery pack. A pressure sensor, H2 gas sensor, and/or CO and/or CO2 gas sensor may be attached inside of a battery pack or battery module.

(A6) In some embodiments of any of A1-A5, the battery system further includes one or more additional sensors configured to a temperature, voltage, and/or current of the one or more battery packs. For example, the one or more additional sensors may include a temperature sensor (e.g., the temperature sensor 126), a pressure sensor (e.g., the pressure sensor 134), a voltage sensor (e.g., the voltage/current sensor 124), and/or a current sensor (e.g., the voltage/current sensor 124). In some embodiments, the one or more additional sensors are used for cell balancing (e.g., the cell balancing 141 in FIG. 1B), battery life estimation (e.g., the remaining useful life 150), determining battery state of health (SoH) (e.g., the SoH 145), determining battery state of charge (SoC) (e.g., the SoC 147), and/or detecting battery abnormalities.

(A7) In some embodiments of any of A1-A6, the control circuitry is further configured to activate an alarm condition based on data from the one or more second sensors. For example, the control circuitry may activate the alarm condition in conjunction with activating the first sensor. In some embodiments, the control circuitry is configured to active the alarm condition in accordance with a determination that the one or more properties meet one or more first criteria. In some embodiments, the control circuitry is configured to active the first sensor in accordance with a determination that the one or more properties meet one or more second criteria. For example, a first level of H2 gas causes the first sensor to be activated and a second level of H2 gas (e.g., greater than the first level) causes the alarm condition to be activated. In some embodiments, activating the alarm condition comprises disabling the one or more battery packs (e.g., using the protection component 160).

(A8) In some embodiments of any of A1-A7, the control circuitry includes a first controller configured to analyze data from the one or more second sensors and a second controller configured to analyze data from the first sensor. In some embodiments, activating the first sensor comprises activating the second controller to analyze the data from the first sensor. In some embodiments, the control circuitry includes a same controller configured to analyze data from the one or more second sensors and analyze data from the first sensor.

(A9) In some embodiments of any of A1-A8, the control circuitry is further configured to activate an alarm condition based on data from the first sensor. For example, the control circuitry may activate the alarm condition in conjunction with disabling the one or more battery packs. In some embodiments, the alarm condition comprises a warning (e.g., an audible and/or visual warning) to move away from the one or more battery packs.

(A10) In some embodiments of any of A1-A9, the control circuitry is configured to identify the increased risk of thermal runaway prior to anode combustion of the one or more battery packs. For example, the control circuitry is configured to detect the thermal runaway condition before significant heating (e.g., in excess of normal operation temperatures) occurs.

(A11) In some embodiments of any of A1-A10, the one or more second properties comprise a presence of hydrogen gas and/or a deformation of at least one of the one or more battery packs.

(A12) In some embodiments of any of A1-A11, detecting the potential risk of thermal runaway in the one or more battery packs comprises detecting electrolysis occurring due to intrusion into at least one of the one or more battery packs. For example, the intrusion may be water intrusion. In some embodiments, the intrusion is a liquid intrusion.

(A13) In some embodiments of any of A1-A12, the increased risk of thermal runaway is identified based on the detection of the first property and the one or more second properties. In some embodiments, the increased risk of thermal runaway is identified based on the one or more second properties without regard for the detection of the first property. For example, in some circumstances the control circuitry may identify the increased risk of thermal runaway without enabling the first sensor. As a specific example, when the one or more second properties indicate a potential risk of thermal runaway, the first sensor is activated to confirm, and when the one or more second properties indicate an imminent thermal runaway, the battery system is disabled without enabling the first sensor and/or without regard to data from the first sensor.

(A14) In some embodiments of any of A1-A13, disabling the battery system comprises disconnecting the one or more battery packs from an electrical circuit. For example, a protection relay (e.g., the protection component 160) is used to disconnect the one or more battery packs.

(A15) In some embodiments of any of A1-A14, detecting the risk of thermal runaway in the one or more battery packs comprises comparing values of the one or more second properties with one or more baseline values. In some embodiments, the baseline values are based on a current state of the one or more battery packs. In some embodiments, the baseline values are based on an initial state of the one or more battery packs (e.g., a state of the battery packs determined during manufacturing).

(A16) In some embodiments of any of A1-A15, the control circuitry is further configured to detect excessive heating and/or overcharging of the one or more battery packs.

(A17) In some embodiments of any of A1-A16, the one or more battery packs include a plurality of lithium-ion battery cells. In some embodiments, each battery pack includes one or more battery cells.

(A18) In some embodiments of any of A1-A17, the battery system is configured for use in a battery electric vehicle. In some embodiments, the battery system is configured for use in a hybrid or EV vehicle.

(A19) In some embodiments of any of A1-A18, the one or more second sensors comprise an accelerometer (e.g., the accelerometer 128) configured to detect an external shock to the battery system. For example, the accelerometer may be a single axis, double axis, or triple axis accelerometer.

(B1) In another aspect, some embodiments include a battery module (e.g., the battery module 110) that includes: (i) one or more battery cells (e.g., the battery cell 112); (ii) a first sensor (e.g., the sensor 136) configured to detect a first property of the battery module; and (iii) one or more second sensors (e.g., one or more of the sensors 124, 126, 128, 130, 132, and 134) coupled to the one or more battery cells of the battery module and configured to detect one or more second properties of the battery module, where the first sensor is selectively activated based on data from the one or more second sensors. In some embodiments, the first sensor is a CO and/or CO2 gas sensor. In some embodiments, the battery module is configured to detect a potential risk of thermal runaway based on data from the first sensor and/or the one or more second sensors. For example, the battery module may include control circuitry configured to detect the potential risk of thermal runaway. In some embodiments, detecting the potential risk of thermal runaway comprises detecting the presence of CO (and/or CO2) within the battery module.

(C1) In yet another aspect, some embodiments include a battery system that includes: (i) two or more of the battery modules of B1; and (ii) control circuitry configured to: (a) identify a potential risk of thermal runaway in a first battery module of the two or more battery modules based on data from the respective one or more second sensors of the first battery module; (b) in response to identifying the potential risk of thermal runaway, activate the respective first sensor of the first battery module; (c) identify an increased risk of thermal runaway based on data from the respective first sensor; and (d) in response to identifying the increased risk of thermal runaway, disable the first battery module.

It will be understood that, although the terms first, second, etc. may be used herein to describe various elements, these elements should not be limited by these terms. These terms are only used to distinguish one element from another. For example, a first array could be termed a second array, and, similarly, a second array could be termed a first array, without departing from the scope of the various described embodiments. The first array and the second array are both arrays, but they are not the same array.

The terminology used in the description of the embodiments herein is for the purpose of describing particular embodiments only and is not intended to be limiting of the scope of claims. As used in the description and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Reference has been made to embodiments, examples of which are illustrated in the accompanying drawings. In the forgoing description, numerous specific details have been set forth in order to provide a thorough understanding of the various described embodiments. However, it will be apparent to one of ordinary skill in the art that the various described embodiments may be practiced without these particular details. In other instances, methods, procedures, components, and circuits that are well-known to those of ordinary skill in the art are not described in detail so as not to unnecessarily obscure aspects of the embodiments.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. The embodiments were chosen and described in order to best explain the principles of the various described embodiments and their practical applications, to thereby enable others skilled in the art to best utilize the principles and the various described embodiments with various modifications as are suited to the particular use contemplated.

What is claimed is:

1. A battery system, comprising:
one or more battery packs;
a first sensor configured to detect a first property of the battery system;
one or more second sensors coupled to the one or more battery packs and configured to detect one or more second properties of the battery system; and
control circuitry coupled to the one or more second sensors and configured to:
detect potential risk of thermal runaway of the one or more battery packs based on the detection of the one or more second properties;
in response to detecting the potential risk of thermal runaway, activate the first sensor;
identify an increased risk of thermal runaway based on detection of the first property of the battery system; and
in response to identifying the increased risk of thermal runaway, disable the battery system.

2. The battery system of claim 1, wherein the one or more second sensors comprise one or more of a hydrogen gas sensor, and a strain sensor.

3. The battery system of claim 1, wherein the one or more second sensors comprise an accelerometer configured to detect an external shock to the battery system.

4. The battery system of claim 1, wherein:
the one or more second sensors comprises a plurality of different types of sensors; and
the control circuitry is configured to detect the potential risk of thermal runaway using a machine learning algorithm to analyze data from the plurality of different types of sensors.

5. The battery system of claim 1, wherein at least one of the one or more second sensors is positioned within a battery pack of the one or more battery packs.

6. The battery system of claim 1, further comprising one or more additional sensors configured to a temperature, voltage, and/or current of the one or more battery packs.

7. The battery system of claim 1, wherein the control circuitry is further configured to activate an alarm condition based on data from the one or more second sensors.

8. The battery system of claim 1, wherein the control circuitry comprises a first controller configured to analyze data from the one or more second sensors and a second controller configured to analyze data from the first sensor.

9. The battery system of claim 1, wherein the control circuitry is further configured to activate an alarm condition based on data from the first sensor.

10. The battery system of claim 1, wherein the control circuitry is configured to identify the increased risk of thermal runaway prior to anode combustion of the one or more battery packs.

11. The battery system of claim 1, wherein the one or more second properties comprise a presence of hydrogen gas and/or a deformation of at least one of the one or more battery packs.

12. The battery system of claim 1, wherein detecting the risk of thermal runaway in the one or more battery packs comprises detecting electrolysis occurring due to intrusion into at least one of the one or more battery packs.

13. The battery system of claim 1, wherein the increased risk of thermal runaway is identified based on the detection of the first property and the one or more second properties.

14. The battery system of claim 1, wherein disabling the battery system comprises disconnecting the one or more battery packs from an electrical circuit.

15. The battery system of claim 1, wherein detecting the risk of thermal runaway in the one or more battery packs comprises comparing values of the one or more second properties with one or more baseline values.

16. The battery system of claim 1, wherein the control circuitry is further configured to detect excessive heating and/or overcharging of the one or more battery packs.

17. The battery system of claim 1, wherein the one or more battery packs comprise lithium-ion battery cells.

18. The battery system of claim 1, wherein the battery system is configured for use in a battery electric vehicle.

19. A battery module, comprising:
one or more battery cells;
a first sensor configured to detect a first property of the battery module; and
one or more second sensors coupled to the one or more battery cells of the battery module and configured to detect one or more second properties of the battery module, wherein the first sensor is selectively activated based on data from the one or more second sensors.

20. A battery system, comprising:
two or more of the battery modules of claim 19; and
control circuitry configured to:
identify a potential risk of thermal runaway in a first battery module of the two or more battery modules based on data from the respective one or more second sensors of the first battery module;
in response to identifying the potential risk of thermal runaway, activate the respective first sensor of the first battery module;
identify an increased risk of thermal runaway based on data from the respective first sensor; and
in response to identifying the increased risk of thermal runaway, disable the first battery module.
